(19) United States
(12) Patent Application Publication
(10) Pub. No.: US 2024/0252514 A1
(43) Pub. Date: Aug. 1, 2024

(54) NEUROACTIVE STEROID FOR THE TREATMENT OF MILD COGNITIVE IMPAIRMENT ASSOCIATED WITH PARKINSON'S DISEASE

(71) Applicant: SAGE THERAPEUTICS, INC., Cambridge, MA (US)

(72) Inventors: James J. Doherty, Cambridge, MA (US); Michael C. Quirk, Cambridge, MA (US); Aaron Michael Koenig, Cambridge, MA (US); Jason Karl Johannesen, Cambridge, MA (US)

(21) Appl. No.: 18/289,550

(22) PCT Filed: May 4, 2022
(86) PCT No.: PCT/US2022/027701

§ 371 Date: Nov. 3, 2023

Related U.S. Application Data
(60) Provisional application No. 63/183,968, filed on May 4, 2021.

(51) Int. Cl. A61K 31/575; A61P 25/16; A61P 25/28

(52) U.S. Cl. CPC A61K 31/575; A61P 25/16; A61P 25/28

(57) ABSTRACT

Provided herein are methods for improving working memory and/or executive function in a subject suffering from Parkinson's Disease, comprising administering to the subject a compound having the formula.

This application claims the benefit of and priority from U.S. Provisional Application No. 63/183,968, filed May 4, 2021, the entire disclosure of which is incorporated herein by reference in its entirety.

### BACKGROUND

Mild cognitive impairment is common in patients who have Parkinson's Disease (PD), and is associated with decreased quality of life and impaired functioning. Patients with PD mild cognitive impairment (PD-MCI) are at a high risk for developing dementia (Litvan et al., MDS Task Force on mild cognitive impairment in Parkinson's disease: critical review of PD-MCI. Mov Disord. 2011; 26(10):1814-1824).

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca²⁺ after binding of the neurotransmitters glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. Positive modulators may be useful as therapeutic agents with potential clinical uses as cognitive enhancers and in the treatment of psychiatric disorders in which glutamatergic transmission is reduced or defective (see, e.g., Horak et al., J. Neuroscience, 2004, 24(46), 10318-10325).

There remains a need for therapeutic agents that treat mild cognitive impairment associated with Parkinson's disease.

### SUMMARY

Provided herein are methods of treating Mild Cognitive Impairment (MCI) associated with Parkinson's Disease comprising administering to the subject a compound having the formula

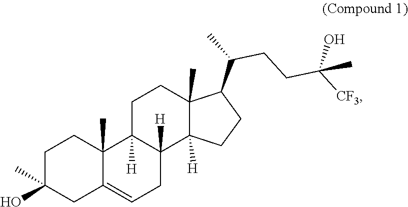

or a pharmaceutically acceptable salt thereof.

Thus, in one aspect, provided herein is a method of treating Mild Cognitive Impairment (MCI) associated with Parkinson's Disease in a subject, the method comprising administering to the subject Compound 1, or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving executive function in a subject having Mild Cognitive Impairment (MCI) associated with Parkinson's Disease, the method comprising administering to the subject Compound 1, or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving working memory in a subject having Mild Cognitive Impairment (MCI) associated with Parkinson's Disease, the method comprising administering to the subject Compound 1, or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning in a subject having Mild Cognitive Impairment (MCI) associated with Parkinson's Disease, the method comprising administering to the subject Compound 1, or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning and working memory in a subject having Mild Cognitive Impairment (MCI) associated with Parkinson's Disease, the method comprising administering to the subject Compound 1, or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In some embodiments, subject is an adult human. In some embodiments, Compound 1 or a pharmaceutically acceptable salt thereof is administered as a monotherapy

### BRIEF DESCRIPTION OF THE DRAWINGS

FIG. 1 depicts an overview of the study design.

FIG. 2 depicts the results of five unique tests of Executive Functioning (MTT, SOC, SWM, 2-Back and DSS) in subjects having Mild Cognitive Impairment associated with Parkinson's Disease (PD-MCI) treated with Compound 1. The tests of Executive Functioning suggest improved performance in PD-MCI subjects treated with Compound 1.

FIG. 3 depicts a detailed analysis of the results of the DSST in PD-MCI subjects treated with Compound 1. An ˜27% improvement over baseline was observed on Treatment Day 14.

FIG. 4 depicts the results of three unique tests of Learning and Memory in PD-MCI subjects treated with Compound 1. These tests suggest improved performance in PD-MCI subjects treated with Compound 1.

FIG. 5 depicts a comparison of Paired Associative Learning in healthy volunteers (Cogstate) and Parkinson's Disease (Cantab) subjects treated Compound 1.

FIG. 6 depicts the results of tests of Attention and Psychomotor Speed in PD-MCI subjects treated with Compound 1. These tests show no appreciable change in PD-MCI subjects treated with Compound 1.

FIG. 7 depicts the results of tests of Social-Emotional Cognition in PD-MCI subjects treated with Compound 1.

### DETAILED DESCRIPTION

As generally described herein, the present invention provides compounds and compositions useful for improving cognitive function in a subject in need thereof.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with any one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

Unless otherwise required by context, singular terms shall include pluralities and plural terms shall include the singular.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

All methods described herein can be performed in any suitable order unless otherwise indicated herein or otherwise clearly contradicted by context.

The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

As used herein, the term “unit dosage form” is defined to refer to the form in which a compound as disclosed herein (e.g., Compound 1) is administered to the subject.

As used herein, the term “daily” means a given, continuous twenty-four (24) hour period.

The terms “subject” and “patient” are used interchangeably herein and include, but are not limited to, humans and a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human (“human subject”). In certain embodiments, the human subject is an infant, child, or adolescent (“pediatric subject”). In other embodiments, the human subject is a young adult, middle-aged adult or senior adult (“adult subject”). In certain embodiments, the subject is a non-human animal (“non-human subject”).

As used herein, and unless otherwise specified, the terms “treat,” “treating” and “treatment” contemplate an action that occurs while a subject is suffering from the specified disease, disorder, or condition, which reduces the severity of the disease, disorder, or condition, or retards or slows the progression of the disease, disorder, or condition (also, “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, to delay or minimize one or more symptoms associated with the disease, disorder or condition, or to improve cognitive function. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In some embodiments, administering a compound as described herein, or a pharmaceutically acceptable salt thereof, improves cognitive function. In some embodiments, the cognitive function refers to a collection of mental tasks and functions, including but not limited to: psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, the cognitive function is one or more selected from the group consisting of psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, Compound 1 selectively targets working memory, and the improvement thereof. In some embodiments, Compound 1 selectively targets executive performance, and the improvement thereof. Measures of cognitive function include assessment tools designed to measure, for example: (a) general intelligence, (b) nonverbal intelligence, (c) achievement, (d) attention/executive functioning, (e) memory and learning, (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered). Such assessment tools are well-known in the art and include, for example, those assessment tools as described in Example 1 herein.

The term “memory” as used herein is defined as the biological processes of the brain that enable storage and recall of information.

The term “working memory” as used herein is defined as a combination of processes of the brain that provide temporary storage and manipulation of information necessary to perform complex cognitive tasks such as learning and reasoning.

The term “learning” as used herein refers to processes of the brain involved in the acquisition of skill, knowledge and information.

The term “executive function” or “executive performance” as used interchangeably herein, refers to an umbrella term for cognitive processes that regulate, control and manage other cognitive processes, including planning, working memory, attention, problem solving, verbal reasoning, inhibition, mental flexibility, task switching, and initiation and monitoring of actions. The prefrontal areas of the frontal lobe are necessary but not sufficient for carrying out these functions.

The term “improve,” “improving”, or “improvement” or grammatical variations thereof used in relation to working memory or executive function refer to the ability to achieve a measurable increase in performance in relation to tasks used to test working memory or executive function in subjects.

### Pharmaceutical Compositions

In one aspect, the disclosure provides a pharmaceutical composition comprising a compound of the present invention (also referred to as the “active ingredient”), for example Compound 1 or a pharmaceutically acceptable salt thereof, and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the compound or pharmaceutical composition is administered to a subject orally. In some embodiments, the compound or pharmaceutical composition is administered to a subject parenterally. In some embodiments, the compound or pharmaceutical composition is administered to a subject rectally. In some embodiments, the compound or pharmaceutical composition is administered to a subject transdermally. In some embodiments, the compound or pharmaceutical composition is administered to a subject intradermally. In some embodiments, the compound or pharmaceutical composition is administered to a subject intrathecally. In some embodiments, the compound or pharmaceutical composition is administered to a subject subcutaneously. In some embodiments, the compound or pharmaceutical composition is administered to a subject intravenously. In some embodiments, the compound or pharmaceutical composition is administered to a subject intramuscularly. In some embodiments, the compound or pharmaceutical composition is administered to a subject intranasally.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

When used to prevent the onset of a CNS-disorder, the compounds provided herein will be administered to a subject at risk for developing the condition, typically on the advice and under the supervision of a physician, at the dosage levels described herein. Subjects at risk for developing a particular condition generally include those that have a family history of the condition, or those who have been identified by genetic testing or screening to be particularly susceptible to developing the condition.

### Methods of Treatment and Use

Provided herein are methods of treating Mild Cognitive Impairment (MCI) associated with Parkinson's Disease, comprising administering to the subject a compound having the formula

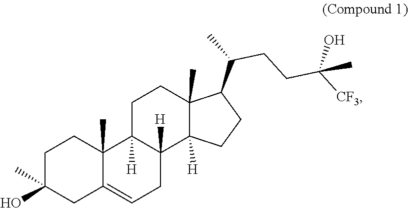

or a pharmaceutically acceptable salt thereof.

Thus, in one aspect, provided herein is a method of treating Mild Cognitive Impairment (MCI) associated with Parkinson's Disease in a subject, the method comprising administering to the subject Compound 1, or a pharmaceutically acceptable salt thereof. In some embodiments, provided herein is a method of treating Mild Cognitive Impairment (MCI) associated with Parkinson's Disease in a subject, the method comprising administering to the subject Compound 1. In some embodiments, provided herein is a method of treating Mild Cognitive Impairment (MCI) associated with Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of treating Mild Cognitive Impairment (MCI) resulting from Parkinson's Disease in a subject, the method comprising administering to the subject Compound 1, or a pharmaceutically acceptable salt thereof. In some embodiments, provided herein is a method of treating Mild Cognitive Impairment (MCI) resulting from Parkinson's Disease in a subject, the method comprising administering to the subject Compound 1. In some embodiments, provided herein is a method of treating Mild Cognitive Impairment (MCI) resulting from Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of treating Mild Cognitive Impairment (MCI) in a subject having Parkinson's Disease, the method comprising administering to the subject Compound 1, or a pharmaceutically acceptable salt thereof. In some embodiments, provided herein is a method of treating Mild Cognitive Impairment (MCI) in a subject having Parkinson's Disease, the method comprising administering to the subject Compound 1. In some embodiments, provided herein is a method of treating Mild Cognitive Impairment (MCI) in a subject having Parkinson's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In one aspect, provided herein is a method of improving executive function in a subject having Mild Cognitive Impairment (MCI) associated with Parkinson's Disease, the method comprising administering to the subject Compound 1, or a pharmaceutically acceptable salt thereof. In some embodiments, provided herein is a method of improving executive function in a subject having Mild Cognitive Impairment (MCI) associated with Parkinson's Disease in a subject, the method comprising administering to the subject Compound 1. In some embodiments, provided herein is a method of improving executive function in a subject having Mild Cognitive Impairment (MCI) associated with Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving executive function in a subject having Mild Cognitive Impairment (MCI) resulting from Parkinson's Disease, the method comprising administering to the subject Compound 1, or a pharmaceutically acceptable salt thereof. In some embodiments, provided herein is a method of improving executive function in a subject having Mild Cognitive Impairment (MCI) resulting from Parkinson's Disease in a subject, the method comprising administering to the subject Compound 1. In some embodiments, provided herein is a method of improving executive function in a subject having Mild Cognitive Impairment (MCI) resulting from Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving executive function in a Parkinson's Disease subject having Mild Cognitive Impairment (MCI), the method comprising administering to the subject Compound 1, or a pharmaceutically acceptable salt thereof. In some embodiments, provided herein is a method of improving executive function, in a Parkinson's Disease subject having Mild Cognitive Impairment (MCI), the method comprising administering to the subject Compound 1. In some embodiments, provided herein is a method of improving executive function in a Parkinson's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In one aspect, provided herein is a method of improving working memory in a subject having Mild Cognitive Impairment (MCI) associated with Parkinson's Disease, the method comprising administering to the subject Compound 1, or a pharmaceutically acceptable salt thereof. In some embodiments, provided herein is a method of improving working memory in a subject having Mild Cognitive Impairment (MCI) associated with Parkinson's Disease in a subject, the method comprising administering to the subject Compound 1. In some embodiments, provided herein is a method of improving working memory in a subject having Mild Cognitive Impairment (MCI) associated with Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving working memory in a subject having Mild Cognitive Impairment (MCI) resulting from Parkinson's Disease, the method comprising administering to the subject Compound 1, or a pharmaceutically acceptable salt thereof. In some embodiments, provided herein is a method of improving working memory in a subject having Mild Cognitive Impairment (MCI) resulting from Parkinson's Disease in a subject, the method comprising administering to the subject Compound 1. In some embodiments, provided herein is a method of improving working memory in a subject having Mild Cognitive Impairment (MCI) resulting from Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving working memory in a Parkinson's Disease subject having Mild Cognitive Impairment (MCI), the method comprising administering to the subject Compound 1, or a pharmaceutically acceptable salt thereof. In some embodiments, provided herein is a method of improving working memory, in a Parkinson's Disease subject having Mild Cognitive Impairment (MCI), the method comprising administering to the subject Compound 1. In some embodiments, provided herein is a method of improving working memory in a Parkinson's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In one aspect, provided herein is a method of improving learning in a subject having Mild Cognitive Impairment (MCI) associated with Parkinson's Disease, the method comprising administering to the subject Compound 1, or a pharmaceutically acceptable salt thereof. In some embodiments, provided herein is a method of improving learning in a subject having Mild Cognitive Impairment (MCI) associated with Parkinson's Disease in a subject, the method comprising administering to the subject Compound 1. In some embodiments, provided herein is a method of improving learning in a subject having Mild Cognitive Impairment (MCI) associated with Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving learning in a subject having Mild Cognitive Impairment (MCI) resulting from Parkinson's Disease, the method comprising administering to the subject Compound 1, or a pharmaceutically acceptable salt thereof. In some embodiments, provided herein is a method of improving learning in a subject having Mild Cognitive Impairment (MCI) resulting from Parkinson's Disease in a subject, the method comprising administering to the subject Compound 1. In some embodiments, provided herein is a method of improving learning in a subject having Mild Cognitive Impairment (MCI) resulting from Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving learning in a Parkinson's Disease subject having Mild Cognitive Impairment (MCI), the method comprising administering to the subject Compound 1, or a pharmaceutically acceptable salt thereof. In some embodiments, provided herein is a method of improving learning in a Parkinson's Disease subject having Mild Cognitive Impairment (MCI), the method comprising administering to the subject Compound 1. In some embodiments, provided herein is a method of improving learning in a Parkinson's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In one aspect, provided herein is a method of improving learning and working memory in a subject having Mild Cognitive Impairment (MCI) associated with Parkinson's Disease, the method comprising administering to the subject Compound 1, or a pharmaceutically acceptable salt thereof. In some embodiments, provided herein is a method of improving learning and working memory in a subject having Mild Cognitive Impairment (MCI) associated with Parkinson's Disease in a subject, the method comprising administering to the subject Compound 1. In some embodiments, provided herein is a method of improving learning and working memory in a subject having Mild Cognitive Impairment (MCI) associated with Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving learning and working memory in a subject having Mild Cognitive Impairment (MCI) resulting from Parkinson's Disease, the method comprising administering to the subject Compound 1, or a pharmaceutically acceptable salt thereof. In some embodiments, provided herein is a method of improving learning and working memory in a subject having Mild Cognitive Impairment (MCI) resulting from Parkinson's Disease in a subject, the method comprising administering to the subject Compound 1. In some embodiments, provided herein is a method of improving learning and working memory in a subject having Mild Cognitive Impairment (MCI) resulting from Parkinson's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In another aspect, provided herein is a method of improving learning and working memory in a Parkinson's Disease subject having Mild Cognitive Impairment (MCI), the method comprising administering to the subject Compound 1, or a pharmaceutically acceptable salt thereof. In some embodiments, provided herein is a method of improving learning and working memory in a Parkinson's Disease subject having Mild Cognitive Impairment (MCI), the method comprising administering to the subject Compound 1. In some embodiments, provided herein is a method of improving learning and working memory in a Parkinson's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally. In some embodiments, the daily dose of Compound 1 or pharmaceutically acceptable salt thereof is administered as a single dose. In some embodiments, the daily dose of Compound 1 or pharmaceutically acceptable salt thereof is administered as multiple doses. In some embodiments, the daily dose of Compound 1 is administered as a single dose. In some embodiments, the daily dose of Compound 1 is administered as multiple doses. In some embodiments, the daily dose of the pharmaceutically acceptable salt of Compound 1 is administered as a single dose. In some embodiments, the daily dose of the pharmaceutically acceptable salt of Compound 1 is administered as multiple doses. In some embodiments, the daily dose of Compound 1 is administered once a day. In some embodiments, the daily dose of Compound 1 is administered as two doses per day. In some embodiments, the daily dose of Compound 1 is administered as three doses per day. In some embodiments, the daily dose of a pharmaceutically acceptable salt of Compound 1 is administered once a day. In some embodiments, the daily dose of a pharmaceutically acceptable salt of Compound 1 is administered as two doses per day. In some embodiments, the daily dose of a pharmaceutically acceptable salt of Compound 1 is administered as three doses per day.

In some embodiments, provided herein is the use of Compound 1, or a pharmaceutically acceptable salt thereof for the manufacture of a medicament for treating Mild Cognitive Impairment (MCI) associated with Parkinson's Disease in a subject. In some embodiments, provided herein is the use of Compound 1 for the manufacture of a medicament for treating Mild Cognitive Impairment (MCI) associated with Parkinson's Disease in a subject. In some embodiments, provided herein is the use of a pharmaceutically acceptable salt of Compound 1 for the manufacture of a medicament for treating Mild Cognitive Impairment (MCI) associated with Parkinson's Disease in a subject. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for treating Mild Cognitive Impairment (MCI) resulting from Parkinson's Disease in a subject. In some embodiments, provided herein is the use of Compound 1 for the manufacture of a medicament for treating Mild Cognitive Impairment (MCI) resulting from Parkinson's Disease in a subject. In some embodiments, provided herein is the use of a pharmaceutically acceptable salt of Compound 1 for the manufacture of a medicament for treating Mild Cognitive Impairment (MCI) resulting from Parkinson's Disease in a subject. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for treating Mild Cognitive Impairment (MCI) in a subject having Parkinson's Disease. In some embodiments, provided herein is the use of Compound 1 for the manufacture of a medicament for treating Mild Cognitive Impairment (MCI) in a subject having Parkinson's Disease. In some embodiments, provided herein is the use of a pharmaceutically acceptable salt of Compound 1 for the manufacture of a medicament for treating Mild Cognitive Impairment (MCI) in a subject having Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving executive function in a subject having Mild Cognitive Impairment (MCI) associated with Parkinson's Disease. In some embodiments, provided herein is the use of Compound 1 for the manufacture of a medicament for improving executive function in a subject having Mild Cognitive Impairment (MCI) associated with Parkinson's Disease. In some embodiments, provided herein is the use of a pharmaceutically acceptable salt of Compound 1 for the manufacture of a medicament for improving executive function in a subject having Mild Cognitive Impairment (MCI) associated with Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving executive function in a subject having Mild Cognitive Impairment (MCI) resulting from Parkinson's Disease. In some embodiments, provided herein is the use of Compound 1 for the manufacture of a medicament for improving executive function in a subject having Mild Cognitive Impairment (MCI) resulting from Parkinson's Disease. In some embodiments, provided herein is the use of a pharmaceutically acceptable salt of Compound 1 for the manufacture of a medicament for improving executive function in a subject having Mild Cognitive Impairment (MCI) resulting from Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving executive function in a Parkinson's Disease subject having Mild Cognitive Impairment (MCI). In some embodiments, provided herein is the use of Compound 1 for the manufacture of a medicament for improving executive function in a Parkinson's Disease subject having Mild Cognitive Impairment (MCI). In some embodiments, provided herein is the use of a pharmaceutically acceptable salt of Compound 1 for the manufacture of a medicament for improving executive function in a Parkinson's Disease subject having Mild Cognitive Impairment (MCI). In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving working memory in a subject having Mild Cognitive Impairment (MCI) associated with Parkinson's Disease. In some embodiments, provided herein is the use of Compound 1 for the manufacture of a medicament for\improving working memory in a subject having Mild Cognitive Impairment (MCI) associated with Parkinson's Disease. In some embodiments, provided herein is the use of a pharmaceutically acceptable salt of Compound 1 for the manufacture of a medicament for\improving working memory in a subject having Mild Cognitive Impairment (MCI) associated with Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving working memory in a subject having Mild Cognitive Impairment (MCI) resulting from Parkinson's Disease. In some embodiments, provided herein is the use of Compound 1 for the manufacture of a medicament for method of improving working memory in a subject having Mild Cognitive Impairment (MCI) resulting from Parkinson's Disease. In some embodiments, provided herein is the use of a pharmaceutically acceptable salt of Compound 1 for the manufacture of a medicament for method of improving working memory in a subject having Mild Cognitive Impairment (MCI) resulting from Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving working memory in a Parkinson's Disease subject having Mild Cognitive Impairment (MCI). In some embodiments, provided herein is the use of Compound 1 for the manufacture of a medicament for improving working memory in a Parkinson's Disease subject having Mild Cognitive Impairment (MCI). In some embodiments, provided herein is the use of a pharmaceutically acceptable salt of Compound 1 for the manufacture of a medicament for improving working memory in a Parkinson's Disease subject having Mild Cognitive Impairment (MCI). In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning in a subject having Mild Cognitive Impairment (MCI) associated with Parkinson's Disease. In some embodiments, provided herein is the use of Compound 1 for the manufacture of a medicament for improving learning in a subject having Mild Cognitive Impairment (MCI) associated with Parkinson's Disease. In some embodiments, provided herein is the use of a pharmaceutically acceptable salt of Compound 1 for the manufacture of a medicament for improving learning in a subject having Mild Cognitive Impairment (MCI) associated with Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning in a subject having Mild Cognitive Impairment (MCI) resulting from Parkinson's Disease. In some embodiments, provided herein is the use of Compound 1 for the manufacture of a medicament for improving learning in a subject having Mild Cognitive Impairment (MCI) resulting from Parkinson's Disease. In some embodiments, provided herein is the use of a pharmaceutically acceptable salt of Compound 1 for the manufacture of a medicament for improving learning in a subject having Mild Cognitive Impairment (MCI) resulting from Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning in a Parkinson's Disease subject having Mild Cognitive Impairment (MCI). In some embodiments, provided herein is the use of Compound 1 for the manufacture of a medicament for improving learning in a Parkinson's Disease subject having Mild Cognitive Impairment (MCI). In some embodiments, provided herein is the use of a pharmaceutically acceptable salt of Compound 1 for the manufacture of a medicament for improving learning in a Parkinson's Disease subject having Mild Cognitive Impairment (MCI). In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning and working memory in a subject having Mild Cognitive Impairment (MCI) associated with Parkinson's Disease. In some embodiments, provided herein is the use of Compound 1 for the manufacture of a medicament for improving learning and working memory in a subject having Mild Cognitive Impairment (MCI) associated with Parkinson's Disease. In some embodiments, provided herein is the use of a pharmaceutically acceptable salt of Compound 1 for the manufacture of a medicament for improving learning and working memory in a subject having Mild Cognitive Impairment (MCI) associated with Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning and working memory in a subject having Mild Cognitive Impairment (MCI) resulting from Parkinson's Disease. In some embodiments, provided herein is the use of Compound 1 for the manufacture of a medicament for improving learning and working memory in a subject having Mild Cognitive Impairment (MCI) resulting from Parkinson's Disease. In some embodiments, provided herein is the use of a pharmaceutically acceptable salt of Compound 1 for the manufacture of a medicament for improving learning and working memory in a subject having Mild Cognitive Impairment (MCI) resulting from Parkinson's Disease. In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is the use of Compound 1, or a pharmaceutically acceptable salt thereof for the manufacture of a medicament for improving learning and working memory in a Parkinson's Disease subject having Mild Cognitive Impairment (MCI). In some embodiments, provided herein is the use of Compound 1 for the manufacture of a medicament for improving learning and working memory in a Parkinson's Disease subject having Mild Cognitive Impairment (MCI). In some embodiments, provided herein is the use of a pharmaceutically acceptable salt of Compound 1 for the manufacture of a medicament for improving learning and working memory in a Parkinson's Disease subject having Mild Cognitive Impairment (MCI). In some embodiments, the medicament is formulated for administering Compound 1 to a subject daily. In some embodiments, the medicament is formulated for administering the pharmaceutically acceptable salt of Compound 1 to a subject daily. In some embodiments, the medicament is formulated for administering Compound 1 or a pharmaceutically acceptable salt thereof to the subject at a dose of about 0.3-6 mg/day. In some embodiments, the medicament is formulated for administering Compound 1 to the subject at a dose of about 0.3-6 mg/day. In some embodiments, the medicament is formulated for administering a pharmaceutically acceptable salt of Compound 1 to the subject at a dose of about 0.3-6 mg/day. In some embodiments, the medicament is formulated for administering Compound 1 or a pharmaceutically acceptable salt thereof to the subject at a dose of about 3 mg/day. In some embodiments, the medicament is formulated for administering Compound 1 to the subject at a dose of about 3 mg/day. In some embodiments, the medicament is formulated for administering a pharmaceutically acceptable salt of Compound 1 to the subject at a dose of about 3 mg/day. In some embodiments, the medicament is formulated for administering Compound 1 or a pharmaceutically acceptable salt thereof to the subject at a dose of 0.3-6 mg/day. In some embodiments, the medicament is formulated for administering Compound 1 to the subject at a dose of 0.3-6 mg/day. In some embodiments, the medicament is formulated for administering a pharmaceutically acceptable salt of Compound 1 to the subject at a dose of 0.3-6 mg/day. In some embodiments, the medicament is formulated for administering Compound 1 or a pharmaceutically acceptable salt thereof to the subject at a dose of 3 mg/day. In some embodiments, the medicament is formulated for administering Compound 1 to the subject at a dose of 3 mg/day. In some embodiments, the medicament is formulated for administering a pharmaceutically acceptable salt of Compound 1 to the subject at a dose of 3 mg/day. In some embodiments, the medicament is formulated for administering Compound 1 orally. In some embodiments, the medicament is formulated for administering a pharmaceutically acceptable salt of Compound 1 orally. In some embodiments, the medicament is formulated for administering the daily dose of Compound 1 or pharmaceutically acceptable salt thereof as a single dose. In some embodiments, the medicament is formulated for administering the daily dose of Compound 1 or pharmaceutically acceptable salt thereof as multiple doses. In some embodiments, the medicament is formulated for administering the daily dose of Compound 1 as a single dose. In some embodiments, the medicament is formulated for administering the daily dose of Compound 1 as multiple doses. In some embodiments, the medicament is formulated for administering the daily dose of the pharmaceutically acceptable salt of Compound 1 as a single dose. In some embodiments, the medicament is formulated for administering the daily dose of the pharmaceutically acceptable salt of Compound 1 as multiple doses. In some embodiments, the medicament is formulated for administering the daily dose of Compound 1 once a day. In some embodiments, the medicament is formulated for administering the daily dose of Compound 1 as two doses per day. In some embodiments, the medicament is formulated for administering the daily dose of Compound 1 as three doses per day. In some embodiments, the medicament is formulated for administering the daily dose of a pharmaceutically acceptable salt of Compound 1 once a day. In some embodiments, the medicament is formulated for administering the daily dose of a pharmaceutically acceptable salt of Compound 1 as two doses per day. In some embodiments, the medicament is formulated for administering the daily dose of a pharmaceutically acceptable salt of Compound 1 as three doses per day.

In some embodiments, provided herein is Compound 1, or a pharmaceutically acceptable salt thereof for use in treating Mild Cognitive Impairment (MCI) associated with Parkinson's Disease in a subject. In some embodiments, provided herein is Compound 1 for use in treating Mild Cognitive Impairment (MCI) associated with Parkinson's Disease in a subject. In some embodiments, provided herein is a pharmaceutically acceptable salt of Compound 1 for use in treating Mild Cognitive Impairment (MCI) associated with Parkinson's Disease in a subject. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in treating Mild Cognitive Impairment (MCI) resulting from Parkinson's Disease in a subject. In some embodiments, provided herein is Compound 1 for use in treating Mild Cognitive Impairment (MCI) resulting from Parkinson's Disease in a subject. In some embodiments, provided herein is a pharmaceutically acceptable salt of Compound 1 for use in treating Mild Cognitive Impairment (MCI) resulting from Parkinson's Disease in a subject. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in treating a Parkinson's Disease subject having Mild Cognitive Impairment (MCI). In some embodiments, provided herein is Compound 1 for use in treating a Parkinson's Disease subject having Mild Cognitive Impairment (MCI). In some embodiments, provided herein is a pharmaceutically acceptable salt of Compound 1 for use in treating a Parkinson's Disease subject having Mild Cognitive Impairment (MCI). In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving executive function in a subject having Mild Cognitive Impairment (MCI) associated with Parkinson's Disease. In some embodiments, provided herein is Compound 1 for use in improving executive function in a subject having Mild Cognitive Impairment (MCI) associated with Parkinson's Disease. In some embodiments, provided herein is a pharmaceutically acceptable salt of Compound 1 for use in improving executive function in a subject having Mild Cognitive Impairment (MCI) associated with Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving executive function in a subject having Mild Cognitive Impairment (MCI) resulting from Parkinson's Disease. In some embodiments, provided herein is Compound 1 for use in improving executive function in a subject having Mild Cognitive Impairment (MCI) resulting from Parkinson's Disease. In some embodiments, provided herein is a pharmaceutically acceptable salt of Compound 1 for use in improving executive function in a subject having Mild Cognitive Impairment (MCI) resulting from Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving executive function in a Parkinson's Disease subject having Mild Cognitive Impairment (MCI). In some embodiments, provided herein is Compound 1 for use in improving executive function in a Parkinson's Disease subject having Mild Cognitive Impairment (MCI). In some embodiments, provided herein is a pharmaceutically acceptable salt of Compound 1 for use in improving executive function in a Parkinson's Disease subject having Mild Cognitive Impairment (MCI). In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving working memory in a subject having Mild Cognitive Impairment (MCI) associated with Parkinson's Disease. In some embodiments, provided herein is Compound 1 for use in improving working memory in a subject having Mild Cognitive Impairment (MCI) associated with Parkinson's Disease. In some embodiments, provided herein is a pharmaceutically acceptable salt of Compound 1 for use in improving working memory in a subject having Mild Cognitive Impairment (MCI) associated with Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving working memory in a subject having Mild Cognitive Impairment (MCI) resulting from Parkinson's Disease. In some embodiments, provided herein is Compound 1 for use in improving working memory in a subject having Mild Cognitive Impairment (MCI) resulting from Parkinson's Disease. In some embodiments, provided herein is a pharmaceutically acceptable salt of Compound 1 for use in improving working memory in a subject having Mild Cognitive Impairment (MCI) resulting from Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving working memory in a Parkinson's Disease subject having Mild Cognitive Impairment (MCI). In some embodiments, provided herein is Compound 1 for use in improving working memory in a Parkinson's Disease subject having Mild Cognitive Impairment (MCI). In some embodiments, provided herein is a pharmaceutically acceptable salt of Compound 1 for use in improving working memory in a Parkinson's Disease subject having Mild Cognitive Impairment (MCI). In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning in a subject having Mild Cognitive Impairment (MCI) associated with Parkinson's Disease. In some embodiments, provided herein is Compound 1 for use in improving learning in a subject having Mild Cognitive Impairment (MCI) associated with Parkinson's Disease. In some embodiments, provided herein is a pharmaceutically acceptable salt of Compound 1 for use in improving learning in a subject having Mild Cognitive Impairment (MCI) associated with Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning in a subject having Mild Cognitive Impairment (MCI) resulting from Parkinson's Disease. In some embodiments, provided herein is Compound 1 for use in improving learning in a subject having Mild Cognitive Impairment (MCI) resulting from Parkinson's Disease. In some embodiments, provided herein is a pharmaceutically acceptable salt of Compound 1 for use in improving learning in a subject having Mild Cognitive Impairment (MCI) resulting from Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning in a Parkinson's Disease subject having Mild Cognitive Impairment (MCI). In some embodiments, provided herein is Compound 1 for use in improving learning in a Parkinson's Disease subject having Mild Cognitive Impairment (MCI). In some embodiments, provided herein is a pharmaceutically acceptable salt of Compound 1 for use in improving learning in a Parkinson's Disease subject having Mild Cognitive Impairment (MCI). In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning and working memory in a subject having Mild Cognitive Impairment (MCI) associated with Parkinson's Disease. In some embodiments, provided herein is Compound 1 for use in improving learning and working memory in a subject having Mild Cognitive Impairment (MCI) associated with Parkinson's Disease. In some embodiments, provided herein is a pharmaceutically acceptable salt of Compound 1 for use in improving learning and working memory in a subject having Mild Cognitive Impairment (MCI) associated with Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning and working memory in a subject having Mild Cognitive Impairment (MCI) resulting from Parkinson's Disease. In some embodiments, provided herein is Compound 1 for use in improving learning and working memory in a subject having Mild Cognitive Impairment (MCI) resulting from Parkinson's Disease. In some embodiments, provided herein is a pharmaceutically acceptable salt of Compound 1 for use in improving learning and working memory in a subject having Mild Cognitive Impairment (MCI) resulting from Parkinson's Disease. In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, provided herein is Compound 1, or a pharmaceutically acceptable salt thereof for use in improving learning and working memory in a Parkinson's Disease subject having Mild Cognitive Impairment (MCI). In some embodiments, provided herein is Compound 1 for use in improving learning and working memory in a Parkinson's Disease subject having Mild Cognitive Impairment (MCI). In some embodiments, provided herein is a pharmaceutically acceptable salt of Compound 1 for use in improving learning and working memory in a Parkinson's Disease subject having Mild Cognitive Impairment (MCI). In some embodiments, Compound 1 is formulated for administration to a subject daily. In some embodiments, the pharmaceutically acceptable salt of Compound 1 is formulated for administration to a subject daily. In some embodiments, Compound 1 or a pharmaceutically acceptable salt thereof is formulated for administration to the subject at a dose of about 0.3-6 mg/day. In some embodiments, Compound 1 is formulated for administration to the subject at a dose of about 0.3-6 mg/day. In some embodiments, a pharmaceutically acceptable salt of Compound 1 is formulated for administration to the subject at a dose of about 0.3-6 mg/day. In some embodiments, Compound 1 or a pharmaceutically acceptable salt thereof is formulated for administration to the subject at a dose of about 3 mg/day. In some embodiments, Compound 1 is formulated for administration to the subject at a dose of about 3 mg/day. In some embodiments, a pharmaceutically acceptable salt of Compound 1 is formulated for administration to the subject at a dose of about 3 mg/day. In some embodiments, Compound 1 or a pharmaceutically acceptable salt thereof is formulated for administration to the subject at a dose of 0.3-6 mg/day. In some embodiments, Compound 1 is formulated for administration to the subject at a dose of 0.3-6 mg/day. In some embodiments, a pharmaceutically acceptable salt of Compound 1 is formulated for administration to the subject at a dose of 0.3-6 mg/day. In some embodiments, Compound 1 or a pharmaceutically acceptable salt thereof is formulated for administration to the subject at a dose of 3 mg/day. In some embodiments, Compound 1 is formulated for administration to the subject at a dose of 3 mg/day. In some embodiments, a pharmaceutically acceptable salt of Compound 1 is formulated for administration to the subject at a dose of 3 mg/day. In some embodiments, Compound 1 is formulated for administration orally. In some embodiments, a pharmaceutically acceptable salt of Compound 1 is formulated for administration orally. In some embodiments, the daily dose of Compound 1 or pharmaceutically acceptable salt thereof is formulated for administration as a single dose. In some embodiments, the daily dose of Compound 1 or pharmaceutically acceptable salt thereof is formulated for administration as multiple doses. In some embodiments, Compound 1 is formulated for administration as a single dose. In some embodiments, Compound 1 is formulated for administration as multiple doses. In some embodiments, the pharmaceutically acceptable salt of Compound 1 is formulated for administration as a single dose. In some embodiments, the pharmaceutically acceptable salt of Compound 1 is formulated for administration as multiple doses. In some embodiments, the daily dose of Compound 1 is formulated for administration once a day. In some embodiments, the daily dose of Compound 1 is formulated for administration as two doses per day. In some embodiments, the daily dose of Compound 1 is formulated for administration as three doses per day. In some embodiments, the daily dose of a pharmaceutically acceptable salt of Compound 1 is formulated for administration once a day. In some embodiments, the daily dose of a pharmaceutically acceptable salt of Compound 1 is formulated for administration as two doses per day. In some embodiments, the daily dose of a pharmaceutically acceptable salt of Compound 1 is formulated for administration as three doses per day.

In some embodiments, the subject having PD-MCI (Mild Cognitive Impairment (MCI) associated with and/or resulting from Parkinson's Disease) (a) has a confirmed diagnosis of idiopathic PD according to 2015 MDS clinical diagnostic criteria (Postuma R B et al. MDS clinical diagnostic criteria for Parkinson's disease. Mov Disord. 2015 October; 30(12):1591-601); and (b) meets the MDS Task Force Criteria for MCI in PD (excluding requirement for UK PD Brain Bank diagnostic criteria) (Litvan et al. Diagnostic criteria for mild cognitive impairment in Parkinson's disease: Movement Disorder Society Task Force guidelines. Mov Disord. 2012 March; 27(3):349-56). In some embodiments, the subject having PD-MCI (Mild Cognitive Impairment (MCI) associated with and/or resulting from Parkinson's Disease has a score of 20-25 (inclusive) on the Montreal Cognitive Assessment (MoCA) (www.mocatest.org/). The Montreal Cognitive Assessment is designed specifically to detect MCI and is widely used in clinical settings. The test is scored from zero to 30, with scores 26 or higher indicating normal cognition (Nasreddine et al; The Montreal Cognitive Assessment, MoCA: A Brief Screening Tool for Mild Cognitive Impairment. J Am Ger Soc. 2005 March; 53(4):695-699).

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

### Example 1: Effect of Compound 1 on Mild Cognitive Impairment Due to Parkinson's Disease

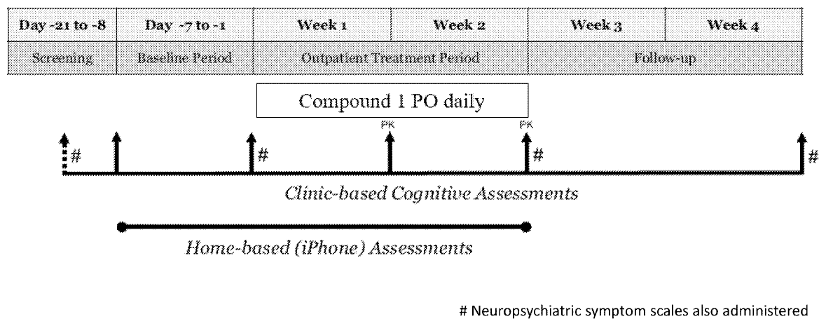

The safety and tolerability of Compound 1 and its effects on cognitive, neuropsychiatric, and motor symptoms in patients suffering from Mild Cognitive Impairment due to Parkinson's Disease (PD-MCI) was evaluated in an open label study. Eligible participants with a confirmed diagnosis of idiopathic PD by 2015 Movement Disorder Society (MDS) criteria (Berardelli et al., EFNS/MDS-ES/ENS recommendations for the diagnosis of Parkinson's disease. Eur J Neurol. 2013; 20:16-34) at Screening and who met MDS Task Force Criteria for MCI in PD (excluding requirement for UK PD Brain Bank diagnostic criteria) received 3.0 mg of Compound 1 (oral dose) daily for 14 days. Participants were instructed to take the daily dose of Compound 1 in the morning within 1 hour of initiating a meal containing approximately 30 g of fat. The study protocol was as follows and is depicted in FIG. 1.

### Screening Period

The Screening Period began with the informed consent process for prospective participants, including study partners. Subsequent screening assessments were performed between Day −21 and Day −8 to determine eligibility, including assessments of cognitive and motor function.

### Baseline Period

The Baseline Period was from Day −7 through Day −1. During this period, each participant received dietary counseling to aid in choosing a morning meal containing approximately 30 g fat to consume prior to dosing. On Day −7 (±1 day), participants will visit the clinic for confirmation of continued eligibility and collection of baseline cognitive and safety data. Additionally, participants were trained on the use of a mobile data capture device to perform daily cognitive tasks and to document sleep patterns. Participants continued to complete daily assessments of cognitive symptoms and sleep using the mobile device at home during the Baseline Period. On Day −1, participants returned to the study site for confirmation of continued eligibility and collection of baseline cognitive and safety data.

### Treatment Period

Beginning on Day 1 and continuing through Day 14, participants self-administered 3.0 mg Compound 1 (as six 0.5-mg oral tablets), once per day in the morning within 1 hour after initiating a meal containing approximately 30 g of fat, in accordance with the guidelines provided in a nutritional information brochure. Participants continued to complete daily assessments of cognitive performance and sleep via mobile device either remotely (at home), or in the clinic at scheduled visits and tracked Compound 1 dosing and predose food intake in a participant diary.

At scheduled visits during the Treatment Period, participants completed the mobile device tasks under staff supervision. At scheduled clinic visits during the Treatment Period, safety, efficacy, and PK procedures were performed. In addition to weekly clinic visits, study staff contacted participants by telephone on Day 4 to document any TEAEs and/or changes in concomitant medications.

### Follow-Up Period

Participants were contacted by telephone on Day 21 to document any AEs and/or changes in concomitant medications. Participants returned to the clinic on Day 28 (±2 days) for a follow-up visit.

Eight patients (male and female) aged 50 to 75 years old with mild cognitive impairment due to Parkinson's disease received 3 mg as oral tablets of Compound 1 daily for 2-weeks.

The key inclusion criteria for the subjects were as follows:
Meet the following criteria for PD-MCI: 1) Have a confirmed diagnosis of idiopathic PD according to 2015 Movement Disorder Society (MDS) clinical diagnostic criteria (Postuma R B et al. MDS clinical diagnostic criteria for Parkinson's disease. Mov Disord. 2015 October; 30(12):1591-601); and 2) Meet MDS Task Force Criteria for MCI in PD (Litvan et al. Diagnostic criteria for mild cognitive impairment in Parkinson's disease: Movement Disorder Society Task Force guidelines. Mov Disord. 2012 March; 27(3):349-56).
Have a score of 20 to 25 (inclusive) on the Montreal Cognitive Assessment (MoCA) at Screening.
Meet criteria for Hoehn & Yahr Stage I to III (mild to moderate motor severity) at Screening.
Have stable motor symptoms for at least 4 weeks prior to screening, in the opinion of the investigator.

The key exclusion criteria for the subjects were as follows:
Have a diagnosis of dementia of any etiology, including but not limited to: Dementia associated with PD (probable or possible), Dementia with Lewy Bodies, Alzheimer's Dementia, and Vascular Dementia.
Have any indication of parkinsonism other than idiopathic PD.
In the opinion of the investigator, be experiencing unpredictable fluctuations in motor and/or nonmotor symptoms associated with PD.
Have an ongoing central nervous system condition other than idiopathic PD, including active neurologic and/or nonremitted psychiatric disorders, in the opinion of the investigator.
Have a history of brain surgery, deep brain stimulation, a significant head injury causing loss of consciousness greater than 30 minutes, or hospitalization due to a brain injury.
Have experienced significant psychotic symptoms within the past 3 months, including those associated with PD medications, as determined by the investigator.

The Primary objective of the study was to assess (1) the percentage of Participants With at Least One Treatment-Emergent Adverse Event (TEAE) (Time Frame: Up to Day 28).

The secondary objectives of the study were to assess:
(1) the percentage of Participants With Clinically Significant Changes in Vital Sign Measurements;
(2) Percentage of Participants With Change from baseline in following Assessments:
   executive function (One Touch Stockings of Cambridge; Spatial Working Memory; Multitasking Test; N-Back Test)
   learning and memory (Verbal Recognition Memory; Paired Associates Learning; Pattern Recognition Memory; Spatial Span);
   social cognition (Emotion Recognition Task; Emotion Bias Task);
   psychomotor function (Reaction Time Task; Psychomotor Vigilance Task; Digit Symbol Substitution Test);
   neuropsychiatric symptoms (Neuropsychiatric Inventory; Questionnaire (NPI-Q); Geriatric Depression Scale; Brief Irritability Test; Apathy Inventory; Penn Parkinson's Daily Activity Scale; National Sleep Foundation Sleep Diary);
   Unified Parkinson's Disease Rating Scale (UPDRS) motor assessment;
   Montreal Cognitive Assessment
   Clinical Global Impressions-Severity scores
   Overall scores on Clinical Global Impressions-Improvement scales
   exploratory peripheral biomarkers
(3) Percentage of Participants With Clinically Significant Changes in Electrocardiogram (ECG) Measurements;
(4) Percentage of Participants With Change in Baseline for Responses on the Columbia Suicide Severity Rating Scale (C-SSRS). The C-SSRS scale consists of a baseline evaluation (at screening) that assesses the lifetime experience of participants with suicidal ideation (SI) and suicidal behavior (SB) and a postbaseline evaluation that focuses on suicidality since the last study visit.

Patients were monitored for various cognitive functions during that period.

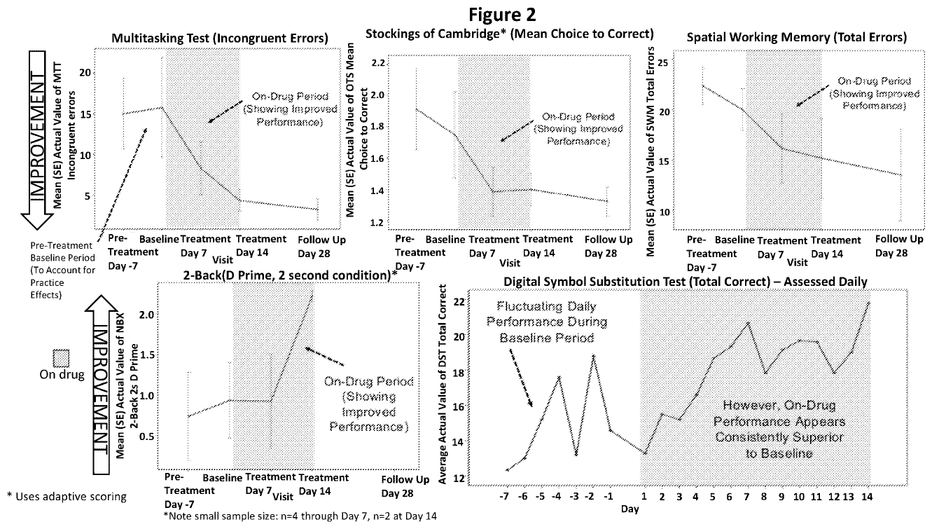

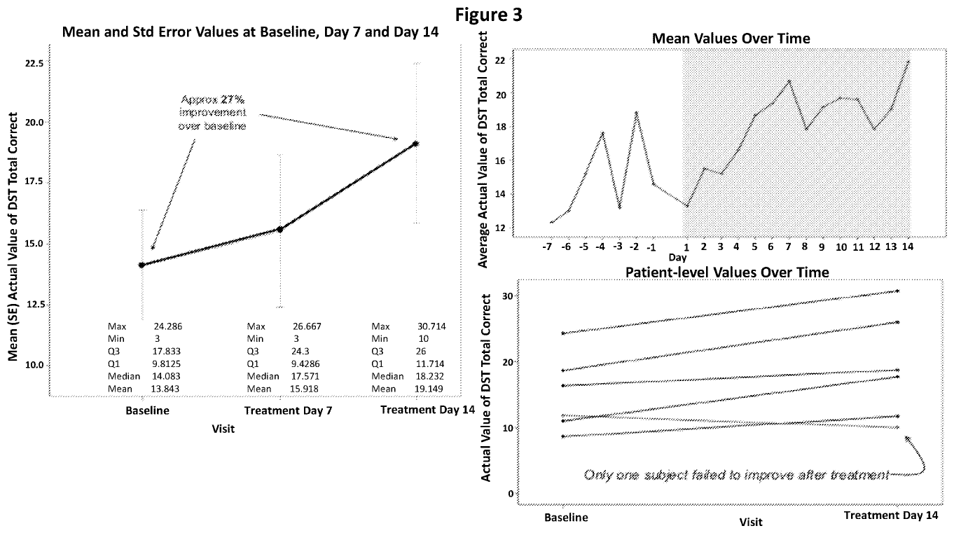

The tests of Executive Functioning included (1) Multitasking Test (MTT), which assesses how well the subject manages conflicting information; (2) One Touch Stockings of Cambridge (OTS), which assesses how well the subject plans, holds onto, and manipulates information to solve a puzzle; (3) Spatial Working Memory (SWM), which assesses how well the subject develops and uses a strategy to find hidden information; (4) Digit Symbol Substitution Task (DSST), which assesses how efficiently can the subject complete a demanding task (daily measure); and (5) Two-Back Test (NBX), which asks is the symbol the same as that shown two symbols ago. Results of these five tests of executive functioning in PD-MCI patients are provided in FIG. 2-3.

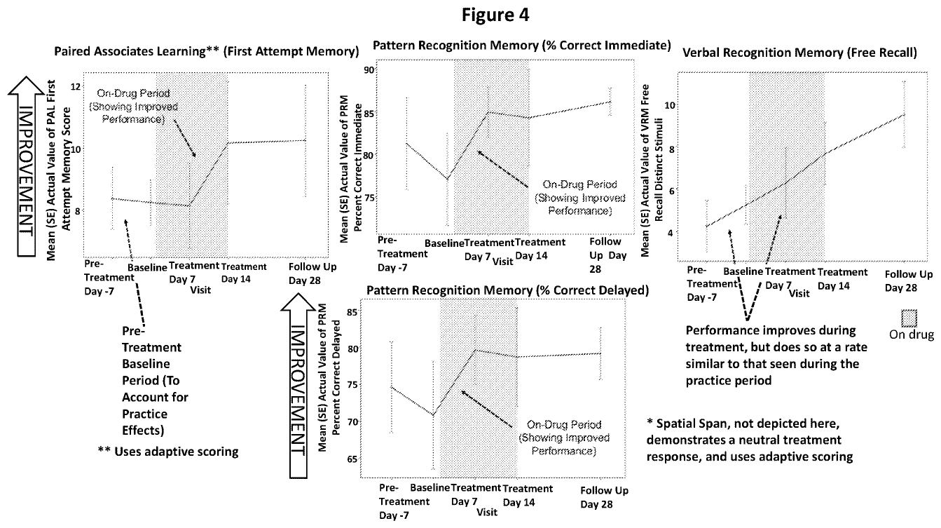

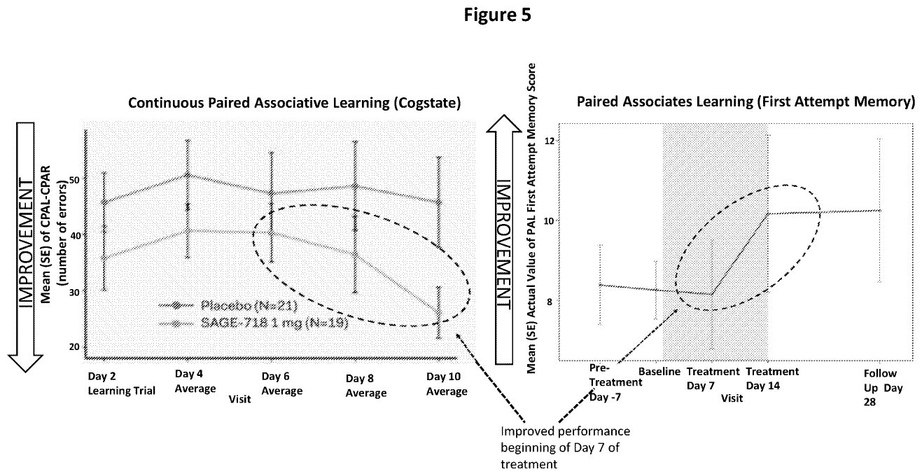

The tests of Learning and Memory included Paired Associates Learning (PAL), which assesses how well the subject remembers the location of hidden objects; Pattern Recognition Memory (PRM), which assesses how well the subject remembers a pattern; and Verbal Recognition Memory (PRM), which assesses how well the subject stores and retrieves verbal information. Results of these tests of learning and memory in PD-MCI patients are provided in FIGS. 4 and 5.

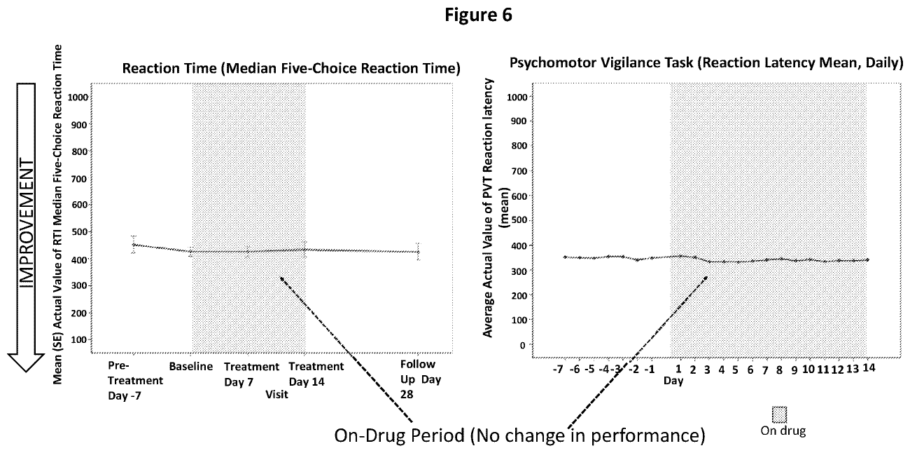

The tests of Attention and Psychomotor Performance included Reaction Time (RTI), which assesses how fast the subject can respond to a stimulus (weekly measure); Psychomotor Vigilance Task (PVT), which assesses how fast can the subject respond to a stimulus (daily measure). Results of these tests of attention and psychomotor performance in PD-MCI patients are provided in FIG. 6.

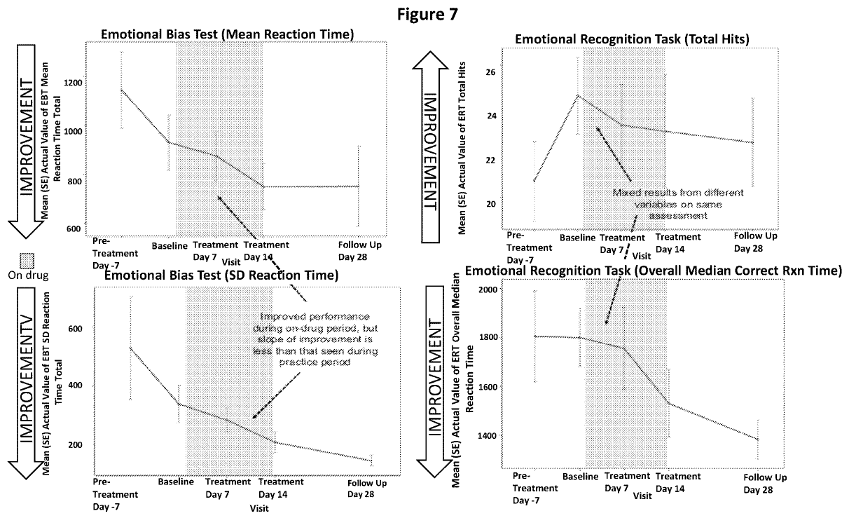

The tests of Social Cognition include Emotion Recognition Task (ERT), which assesses how quickly and accurately can the subject differentiate across a range of emotions; and Emotion Bias Task (EBT), which assesses how quickly and consistently can the subject identify an “ambiguous” emotion. Results of these tests of social cognition in PD-MCI patients are provided in FIG. 7.

As demonstrated in FIGS. 2-3, patients showed performance improvements from baseline across multiple tasks in the cognitive domain of executive function during the 14 days of treatment. Patients also exhibited improved performance from baseline on additional cognitive tests in the domains of learning and memory over a similar timeframe. See FIGS. 4 and 5. Patients exhibited neutral results in certain tests of attention and psychomotor speed. See FIG. 6. Compound 1 was generally well tolerated; there were no serious adverse events and no treatment emergent adverse events were determined to be related to Compound 1.

A summary of the cognitive findings is provided in Table 1:

TABLE 1

| Domain | Task | Setting/Frequency | Trend |
|---|---|---|---|
| Executive | Multitasking | Clinic/Weekly | Improvement |
| Functioning | One Touch Stockings | Clinic/Weekly | Improvement |
| | Spatial working Memory | Clinic/Weekly | Improvement |
| | DSST | Mobile/Daily | Improvement |
| | 2-Back | Clinic/Weekly | Improvement |
| Learning and Memory | Paired Associates | Clinic/Weekly | Improvement |
| | Pattern recognition | Clinic/Weekly | Improvement |
| | Verbal Memory | Clinic/Weekly | Improvement |
| | Spatial Span | Clinic/Weekly | Neutral |
| Attention & | Reaction Time | Clinic/Weekly | Neutral |
| Psychomotor Speed | Psychomotor Vigilance | Mobile/Daily | Neutral |
| Social Cognition | Emotional Bias | Clinic/Weekly | Improvement |
| | Emotional Recognition | Clinic/Weekly | Neutral |

1. A method of treating Mild Cognitive Impairment (MCI) associated with Parkinson's Disease in a subject, comprising administering to the subject a compound having the formula of Compound 1 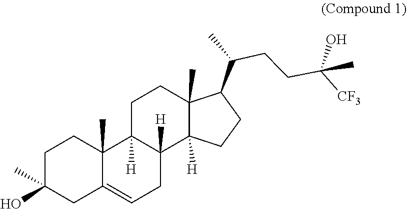
or a pharmaceutically acceptable salt thereof.

2. The method of claim 1, wherein Compound 1 or a pharmaceutically acceptable salt thereof is administered to the subject daily.

3. The method of claim 1 or 2, wherein Compound 1 or a pharmaceutically acceptable salt thereof is administered to the subject at a dose of about 3 mg/day.

4. The method of claim 3, wherein about 3 mg/day of Compound 1 is administered to the subject.

5. The method of claim 3, wherein about 3 mg/day of a pharmaceutically acceptable salt of Compound 1 is administered to the subject.

6. The method of any one of claims 1-5, wherein Compound 1 or a pharmaceutically acceptable salt thereof is administered orally.

7. A method of improving executive function in a subject having Mild Cognitive Impairment (MCI) associated with Parkinson's Disease, the method comprising administering to the subject a compound having the formula of Compound 1 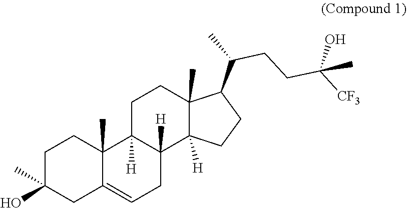
or a pharmaceutically acceptable salt thereof.

8. The method of claim 7, wherein Compound 1 or a pharmaceutically acceptable salt thereof is administered to the subject daily.

9. The method of claim 7 or 8, wherein Compound 1 or a pharmaceutically acceptable salt thereof is administered to the subject at a dose of about 3 mg/day.

10. The method of claim 9, wherein about 3 mg/day of Compound 1 is administered to the subject.

11. The method of claim 9, wherein about 3 mg/day of a pharmaceutically acceptable salt of Compound 1 is administered to the subject.

12. The method of any one of claims 7-11, wherein Compound 1 or a pharmaceutically acceptable salt thereof is administered orally.

13. A method of improving working memory in a subject having Mild Cognitive Impairment (MCI) associated with Parkinson's Disease, the method comprising administering to the subject a compound having the formula of Compound 1 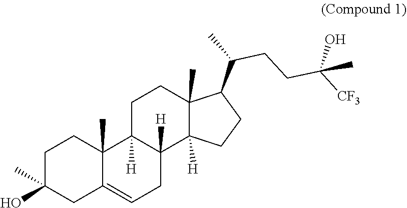
or a pharmaceutically acceptable salt thereof.

14. The method of claim 13, wherein Compound 1 or a pharmaceutically acceptable salt thereof is administered to the subject daily.

15. The method of claim 13 or 14, wherein Compound 1 or a pharmaceutically acceptable salt thereof is administered to the subject at a dose of about 3 mg/day.

16. The method of claim 15, wherein about 3 mg/day of Compound 1 is administered to the subject.

17. The method of claim 15, wherein about 3 mg/day of a pharmaceutically acceptable salt of Compound 1 is administered to the subject.

18. The method of any one of claims 13-17, wherein Compound 1 or a pharmaceutically acceptable salt thereof is administered orally.

19. A method of improving learning in a subject having Mild Cognitive Impairment (MCI) associated with Parkinson's Disease, the method comprising administering to the subject a compound having the formula of Compound 1 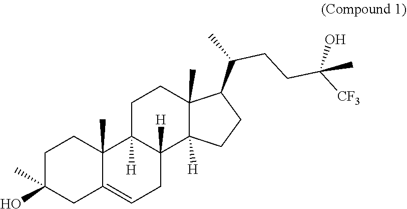
or a pharmaceutically acceptable salt thereof.

20. The method of claim 19, wherein Compound 1 or a pharmaceutically acceptable salt thereof is administered to the subject daily.

21. The method of claim 19 or 20, wherein Compound 1 or a pharmaceutically acceptable salt thereof is administered to the subject at a dose of about 3 mg/day.

22. The method of claim 21, wherein about 3 mg/day of Compound 1 is administered to the subject.

23. The method of claim 21, wherein about 3 mg/day of a pharmaceutically acceptable salt of Compound 1 is administered to the subject.

24. The method of any one of claims 19-23, wherein Compound 1 or a pharmaceutically acceptable salt thereof is administered orally.

25. A method of improving learning and working memory in a subject having Mild Cognitive Impairment (MCI) associated with Parkinson's Disease, the method comprising administering to the subject a compound having the formula of Compound 1 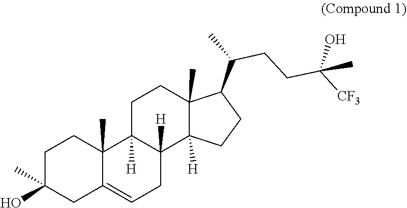
or a pharmaceutically acceptable salt thereof.

26. The method of claim 25, wherein Compound 1 or a pharmaceutically acceptable salt thereof is administered to the subject daily.

27. The method of claim 25 or 26, wherein Compound 1 or a pharmaceutically acceptable salt thereof is administered to the subject at a dose of about 3 mg/day.

28. The method of claim 27, wherein about 3 mg/day of Compound 1 is administered to the subject.

29. The method of claim 27, wherein about 3 mg/day of a pharmaceutically acceptable salt of Compound 1 is administered to the subject.

30. The method of any one of claims 25-29, wherein Compound 1 or a pharmaceutically acceptable salt thereof is administered orally.

31. The method of any one of claims 1-30, wherein the subject is an adult.

32. Use of Compound 1 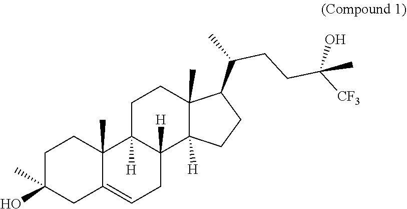
or a pharmaceutically acceptable salt thereof,
for the manufacture of a medicament for treating Mild Cognitive Impairment (MCI) associated with Parkinson's Disease in a subject.

33. The use of claim 32, wherein the medicament is formulated for administering Compound 1 or a pharmaceutically acceptable salt thereof to the subject daily.

34. The use of claim 32 or 33, wherein the medicament is formulated for administering Compound 1 or a pharmaceutically acceptable salt thereof to the subject at a dose of about 3 mg/day.

35. The use of claim 34, wherein the medicament is formulated for administering about 3 mg/day of Compound 1 to the subject.

36. The use of claim 34, wherein the medicament is formulated for administering about 3 mg/day of a pharmaceutically acceptable salt of Compound 1 to the subject.

37. The use of any one of claims 32-36, wherein the medicament is formulated for administering Compound 1 or a pharmaceutically acceptable salt thereof orally.

38. Use of Compound 1 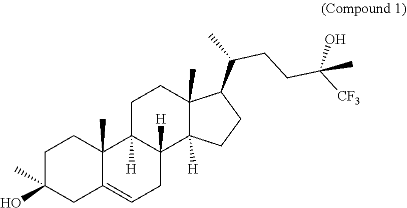
or a pharmaceutically acceptable salt thereof,
for the manufacture of a medicament for improving executive function in a subject having Mild Cognitive Impairment (MCI) associated with Parkinson's Disease.

39. The use of claim 38, wherein the medicament is formulated for administering Compound 1 or a pharmaceutically acceptable salt thereof to the subject daily.

40. The use of claim 38 or 39, wherein the medicament is formulated for administering Compound 1 or a pharmaceutically acceptable salt thereof to the subject at a dose of about 3 mg/day.

41. The use of claim 40, wherein the medicament is formulated for administering about 3 mg/day of Compound 1 to the subject.

42. The use of claim 40, wherein the medicament is formulated for administering about 3 mg/day of a pharmaceutically acceptable salt of Compound 1 to the subject.

43. The use of any one of claims 38-42, wherein the medicament is formulated for administering Compound 1 or a pharmaceutically acceptable salt thereof orally.

44. Use of Compound 1 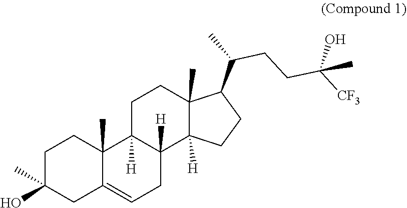
or a pharmaceutically acceptable salt thereof,
for the manufacture of a medicament for improving working memory in a subject having Mild Cognitive Impairment (MCI) associated with Parkinson's Disease.

45. The use of claim 44, wherein the medicament is formulated for administering Compound 1 or a pharmaceutically acceptable salt thereof to the subject daily.

46. The use of claim 44 or 45, wherein the medicament is formulated for administering Compound 1 or a pharmaceutically acceptable salt thereof to the subject at a dose of about 3 mg/day.

47. The use of claim 46, wherein the medicament is formulated for administering about 3 mg/day of Compound 1 to the subject.

48. The use of claim 46, wherein the medicament is formulated for administering about 3 mg/day of a pharmaceutically acceptable salt of Compound 1 to the subject.

49. The use of any one of claims 44-48, wherein the medicament is formulated for administering Compound 1 or a pharmaceutically acceptable salt thereof orally.

50. Use of Compound 1 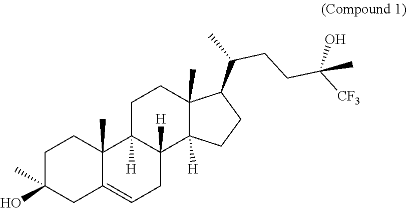
or a pharmaceutically acceptable salt thereof,
for the manufacture of a medicament for improving learning in a subject having Mild Cognitive Impairment (MCI) associated with Parkinson's Disease.

51. The use of claim 50, wherein the medicament is formulated for administering Compound 1 or a pharmaceutically acceptable salt thereof to the subject daily.

52. The use of claim 50 or 51, wherein the medicament is formulated for administering Compound 1 or a pharmaceutically acceptable salt thereof to the subject at a dose of about 3 mg/day.

53. The use of claim 52, wherein the medicament is formulated for administering about 3 mg/day of Compound 1 to the subject.

54. The use of claim 52, wherein the medicament is formulated for administering about 3 mg/day of a pharmaceutically acceptable salt of Compound 1 to the subject.

55. The use of any one of claims 50-54, wherein the medicament is formulated for administering Compound 1 or a pharmaceutically acceptable salt thereof orally.

56. Use of Compound 1 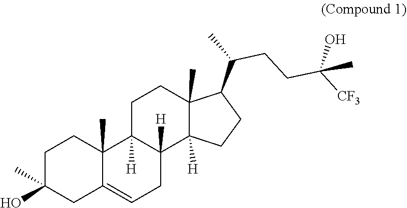
or a pharmaceutically acceptable salt thereof,
for the manufacture of a medicament for improving learning and working memory in a subject having Mild Cognitive Impairment (MCI) associated with Parkinson's Disease.

57. The use of claim 56, wherein the medicament is formulated for administering Compound 1 or a pharmaceutically acceptable salt thereof to the subject daily.

58. The use of claim 56 or 57, wherein the medicament is formulated for administering Compound 1 or a pharmaceutically acceptable salt thereof to the subject at a dose of about 3 mg/day.

59. The use of claim 58, wherein the medicament is formulated for administering about 3 mg/day of Compound 1 to the subject.

60. The use of claim 58, wherein the medicament is formulated for administering about 3 mg/day of a pharmaceutically acceptable salt of Compound 1 to the subject.

61. The use of any one of claims 56-60, wherein the medicament is formulated for administering Compound 1 or a pharmaceutically acceptable salt thereof orally.

62. The use of any one of claims 32-61, wherein the subject is an adult.

63. A compound having the structure of Compound 1 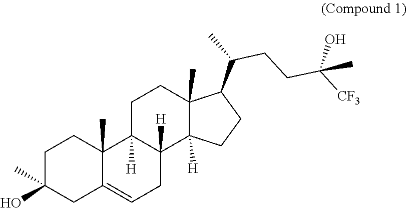
or a pharmaceutically acceptable salt thereof,
for use in treating Mild Cognitive Impairment (MCI) associated with Parkinson's Disease in a subject.

64. The compound for use of claim 63, wherein Compound 1 or a pharmaceutically acceptable salt thereof is formulated for administration to the subject daily.

65. The compound for use of claim 63 or 64, wherein Compound 1 or a pharmaceutically acceptable salt thereof is formulated for administration to the subject at a dose of about 3 mg/day.

66. The compound for use of claim 65, wherein Compound 1 is formulated for administration to the subject at a dose of about 3 mg/day.

67. The compound for use of claim 65, wherein the pharmaceutically acceptable salt of Compound 1 is formulated for administration to the subject at a dose of about 3 mg/day.

68. The compound for use of any one of claims 63-67, wherein Compound 1 or a pharmaceutically acceptable salt thereof is formulated for oral administration.

69. A compound having the structure of Compound 1 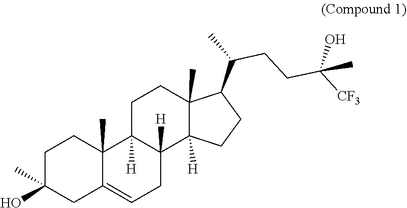
or a pharmaceutically acceptable salt thereof,
for use in improving executive function in a subject having Mild Cognitive Impairment (MCI) associated with Parkinson's Disease.

70. The compound for use of claim 69, wherein Compound 1 or a pharmaceutically acceptable salt thereof is formulated for administration to the subject daily.

71. The compound for use of claim 69 or 70, wherein Compound 1 or a pharmaceutically acceptable salt thereof is formulated for administration to the subject at a dose of about 3 mg/day.

72. The compound for use of claim 71, wherein Compound 1 is formulated for administration to the subject at a dose of about 3 mg/day.

73. The compound for use of claim 71, wherein the pharmaceutically acceptable salt of Compound 1 is formulated for administration to the subject at a dose of about 3 mg/day.

74. The compound for use of any one of claims 69-73, wherein Compound 1 or a pharmaceutically acceptable salt thereof is formulated for oral administration.

75. A compound having the structure of Compound 1 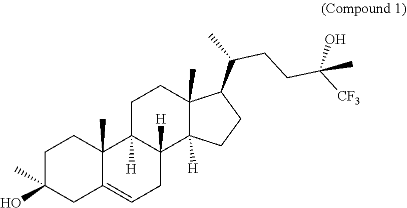
or a pharmaceutically acceptable salt thereof,
for use in improving working memory in a subject having Mild Cognitive Impairment (MCI) associated with Parkinson's Disease.

76. The compound for use of claim 75, wherein Compound 1 or a pharmaceutically acceptable salt thereof is formulated for administration to the subject daily.

77. The compound for use of claim 75 or 76, wherein Compound 1 or a pharmaceutically acceptable salt thereof is formulated for administration to the subject at a dose of about 3 mg/day.

78. The compound for use of claim 77, wherein Compound 1 is formulated for administration to the subject at a dose of about 3 mg/day.

79. The compound for use of claim 77, wherein the pharmaceutically acceptable salt of Compound 1 is formulated for administration to the subject at a dose of about 3 mg/day.

80. The compound for use of any one of claims 75-79, wherein Compound 1 or a pharmaceutically acceptable salt thereof is formulated for oral administration.

81. A compound having the structure of Compound 1 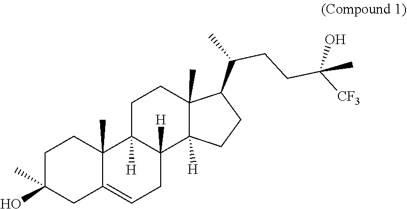
or a pharmaceutically acceptable salt thereof,
for use in improving learning in a subject having Mild Cognitive Impairment (MCI) associated with Parkinson's Disease.

82. The compound for use of claim 81, wherein Compound 1 or a pharmaceutically acceptable salt thereof is formulated for administration to the subject daily.

83. The compound for use of claim 81 or 82, wherein Compound 1 or a pharmaceutically acceptable salt thereof is formulated for administration to the subject at a dose of about 3 mg/day.

84. The compound for use of claim 83, wherein Compound 1 is formulated for administration to the subject at a dose of about 3 mg/day.

85. The compound for use of claim 83, wherein the pharmaceutically acceptable salt of Compound 1 is formulated for administration to the subject at a dose of about 3 mg/day.

86. The compound for use of any one of claims 81-85, wherein Compound 1 or a pharmaceutically acceptable salt thereof is formulated for oral administration.

87. A compound having the structure of Compound 1 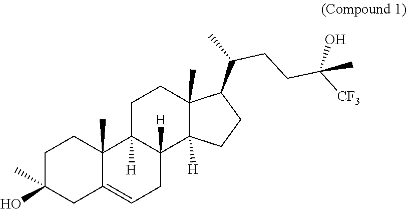
or a pharmaceutically acceptable salt thereof,
for use in improving learning and working memory in a subject having Mild Cognitive Impairment (MCI) associated with Parkinson's Disease.

88. The compound for use of claim 87, wherein Compound 1 or a pharmaceutically acceptable salt thereof is formulated for administration to the subject daily.

89. The compound for use of claim 87 or 88, wherein Compound 1 or a pharmaceutically acceptable salt thereof is formulated for administration to the subject at a dose of about 3 mg/day.

90. The compound for use of claim 89, wherein Compound 1 is formulated for administration to the subject at a dose of about 3 mg/day.

91. The compound for use of claim 89, wherein the pharmaceutically acceptable salt of Compound 1 is formulated for administration to the subject at a dose of about 3 mg/day.

92. The compound for use of any one of claims 87-91, wherein Compound 1 or a pharmaceutically acceptable salt thereof is formulated for oral administration.

93. The use of any one of claims 63-92, wherein the subject is an adult.
